SEQUENCE LISTING (5 sequences)

SEQ ID NO: 1 - DNA, 24 bp, Artificial Sequence
  Synthesized oligonucleotide sequence
gatccttctgggaattcctagatc

SEQ ID NO: 2 - DNA, 21 bp, Artificial Sequence
  Combined DNA/RNA sequence (oligoribonucleotide sequence)
gcagcugcccagcccgccctt

SEQ ID NO: 3 - DNA, 20 bp, Artificial Sequence
  Combined DNA/RNA sequence (oligoribonucleotide sequence)
gggcgggcugggcagcugtt

SEQ ID NO: 4 - DNA, 21 bp, Artificial Sequence
  Combined DNA/RNA sequence (oligoribonucleotide sequence)
cccucccaccauugaacugtt

SEQ ID NO: 5 - DNA, 21 bp, Artificial Sequence
  Combined DNA/RNA sequence (oligoribonucleotide sequence)
caguucaauggugggagggtt

(19) United States
(12) Patent Application Publication
(10) Pub. No.: US 2008/0064027 A1
(43) Pub. Date: Mar. 13, 2008

(54) TEL/Etv6-mediated inhibition of cell proliferation

(76) Inventor: Ali BADACHE, Rixheim (FR)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/533,503

(22) PCT Filed: Nov. 4, 2003
(86) PCT No.: PCT/EP03/12295

§ 371 Date: Oct. 26, 2005

Related U.S. Application Data
(60) Provisional application No. 60/495,309, filed on Aug. 14, 2003.

(30) Foreign Application Priority Data
Nov. 5, 2002 (GB) 0225799.6

(51) Int. Cl. C12Q 1/68; G01N 33/574

(52) U.S. Cl. 435/6; 435/7.23

(57) ABSTRACT

A method of modulating Stat3-dependent and cytokine-sensitive cell proliferation is provided, as well as screening methods for agents affecting Stat3-dependent and cytokine-sensitive cell proliferation. The method is exemplified by modulating TEL/ETV 6.

The invention relates to the field of cell proliferation, with applications in cancer diagnosis and therapy. The invention also relates to the screening of compounds for potential anti-cancer activity, whether prophylactic or therapeutic.

### BACKGROUND OF THE INVENTION

In the more affluent countries of the world cancer is the cause of death of roughly one person in five. The American Cancer Society in 1993 reported that the five most common cancers are those of the lung, stomach, breast, colon/rectum and the uterine cervix. Cancer is not fatal in every case and only about half the number of people who develop cancer die of it. The problem facing cancer patients and their physicians is that seeking to cure cancer is like trying to get rid of weeds. Although cancer cells can be removed surgically or destroyed with toxic compounds or with radiation, it is very hard to eliminate all of the cancerous cells. A general goal is to find better ways of selectively killing cancer cells whilst leaving normal cells of the body unaffected. Part of that effort involves identifying new anti-cancer agents.

Cancer cells have lost the normal control of the cell cycle and so divide out of control compared to normal cells. The sub-cellular machinery which controls the cell cycle is a complex biochemical device made up of a set of interacting proteins that induce and co-ordinate the essential processes of duplication and division of the contents of a cell. In the normal cell cycle, the control system is regulated such that it can stop at specific points in the cycle. The stopping points allow for systems of feedback control from the processes of duplication or division. They also provide points for regulation by environmental signals.

The study of cancer cells has helped to show how growth factors regulate cell proliferation in normal cells through a complex network of intracellular signalling cascades. These cascades ultimately regulate gene transcription and the assembly and activation of the cell cycle control system. As knowledge increases about the component parts of the cell cycle control machinery and how it operates, the possibilities for correcting the loss of control in cancer cells are increased. Essential points of control and essential proteins can be identified in the control hierarchy and potentially targetted with drugs to act as activators or inhibitors, as required.

Signal transducer and activator of transcription 3 (Stat3) mediates the effect of many growth factors and cytokines. While several studies have described the possible role of Stat3 in oncogenesis (see for example, J. Bromberg, 2002, The Journal of Clinical Investigation, 109, 1139-1142; Levy and Lee, 2002, The Journal of Clinical Investigation, 109, 1143-1148), others have shown that Stat3 is in some instances a mediator of cytokine-induced inhibition of tumor cell proliferation (see for example, J. Behrmann, 1999, Oncogene, 18, 3742-53; S Akira, 1996, Proc Natl Acad Sci USA, 93, 3963-3966). Thus, the function of Stat3 in both normal and malignant cells still needs clarification. The presence of inappropriately activated Stats (including Stat 3) in primary tumors and tumor cell lines and their role in aberrant STAT signalling has been reviewed by Bowman et al. (Oncogene, 2000, 19:2474-2488).

The TEL (“translocation-ETS-leukemia”, also known as ETV6) gene is a major target of translocations in leukaemia and its gene product has been described as a putative tumor suppressor in leukemias and some solid tumors. Expression of TEL in Ras-transformed NIH 3T3 cells inhibited cell growth in soft agar and in normal cultures (Fenrick et al., 2000, Molecular and Cellular Biology, 20, 5828-5839). TEL has also been shown to retard tumor formation in nude mice (Van Rompaey et al., 2000, 19, 5244-5250).

### SUMMARY OF THE INVENTION

In accordance with a first aspect of the invention, a method of identifying an agent effective in modulating Stat3-dependent cell proliferation is provided, comprising i) incubating TEL/Etv6 (or a variant or fragment thereof) with a compound; ii) detecting TEL/Etv6 activity; and iii) determining a compound-induced modulation in the TEL/Etv6 activity relative to when the compound is absent, wherein an alteration of the TEL/Etv6 activity in the presence of the compound is indicative of an agent effective in modulating Stat3-dependent cell proliferation.

The modulation can be inhibition of TEL/Etv6 activity, in which case the agent is effective in enhancing cytokine-induced inhibition of cell proliferation. Alternatively, the modulation is activation of TEL/Etv6 activity, in which case the agent is effective in inhibiting proliferation of cells overexpressing Stat 3, in particular phosphorylated Stat3. In the latter case, the cell proliferation can be independent of ras.

Also provided is a method for identifying an agent effective in modulating Stat3-dependent cell proliferation, said method comprising the steps of: (i) incubating a TEL/Etv6 polypeptide selected from: TEL/Etv6, a variant or fragment thereof with a binding partner in the presence of a test compound; and (ii) determining whether the presence of a test compound modulates the interaction between the TEL/Etv6 polypeptide and the binding partner relative to when the test compound is absent. The variant or fragment of TEL/Etv6 preferably has the ability to bind Stat3. The TEL/Etv6 fragment is preferably between 50 and 350 amino acids in length. A preferred binding partner is Stat3, a variant or fragment thereof. Optionally, the TEL/Etv6 polypeptide or the binding partner is labelled with a detectable label, and the other is immobilised on a solid support.

In a further aspect of the invention, the screening methods comprise confirming that the test compound is a modulator of Stat3-dependent cell proliferation.

The modulation may be inhibition of TEL activity or the TEL-binding partner interaction, in which case the method may comprise the step of confirming that the substance inhibits cell proliferation of a cytokine-sensitive cancer cell.

The methods may be in vitro or cell based assays. Thus, also provided by the invention is a mammalian cell capable of expressing a TEL/Etv6 polypeptide, its binding partner, and a reporter gene construct, whereby binding of the TEL/Etv6 polypeptide and the binding partner (in particular Stat3) can be observed by reporter gene expression.

The methods are particularly useful in identifying agents effective in inhibiting cell proliferation of a melanoma or carcinoma.

Also provided are methods of inhibiting Stat3 expressing cancer cell proliferation, in particular melanomas or carcinomas. In one embodiment, the method comprises contacting a cancer cell expressing Stat3, preferably phosphorylated Stat3, with an effective amount of an activator of TEL in an amount sufficient to inhibit Stat3 activity. Preferably, the cell proliferation is independent of ras.

In a further embodiment, the invention provides a method of inhibiting cytokine sensitive cancer cell growth, comprising contacting a cytokine-sensitive cancer cell with an effective amount of a TEL inhibitor in an amount sufficient to enhance Stat3 activity. The TEL inhibitor can be an RNAi or an antibody, for example.

### DETAILED DESCRIPTION OF THE INVENTION

The present invention results from studies on the role of Stat3 in cell proliferation. The present inventors have evaluated the effect of an inducible Stat3 construct in a cytokine-sensitive melanoma cell line and found that activation of Stat3 to moderate levels was sufficient to repress cell proliferation, by slowing down cell transit through the cell cycle (see Examples 1 and 3). Furthermore, enhanced and prolonged Stat3 activity led to cell cycle arrest and apoptosis.

Using oligonucleotide microarray analysis, the present inventors have further identified genes that have increased or decreased expression upon Stat3 activation (see Example 4). One such Stat3 target gene was found to be the transcription factor C/EBPdelta, which is one of the mediators of Stat3 inhibitory activity on cell proliferation, as is supported by targeted disruption of C/EBPdelta expression using small interfering (si) RNA (see Example 5). In contrast, inhibition of expression of another Stat3 target, the TEL/Etv6 transcription factor, increased Stat3 activity further supporting the finding that Stat3 inhibits cell proliferation. Thus, TEL/Etv6 is an inhibitor of Stat3 activity and modulation of TEL/Etv6 allows regulation of the Stat3 signaling pathway (see Example 6).

Accordingly, the present invention provides a method of identifying an agent effective in reducing STAT3-dependent cell proliferation, based on the modulation of TEL/Etv6 levels or TEL/Etv6 activity. Typically such a method will comprise the steps of i) incubating TEL/Etv6 with a compound; ii) detecting the level of TEL/Etv6 or its activity; and iii) determining a compound-induced modulation in the level of TEL/Etv6 or its activity relative to when the compound is absent, wherein an alteration of the level of TEL/Etv6 or its activity in the presence of the compound is indicative of an agent effective in modulating Stat3-dependent cell proliferation.

The terms “TEL” and “Etv6” are used interchangeably herein and refer to ets variant gene 6 (TEL oncogene; NCBI accession number NM_{—}001987; Golub, et al., Cell 77 (2), 307-316, 1994).

As those skilled in the art will appreciate, unless context demands otherwise, where polypeptides or nucleic acids are referred to in aspects and embodiments of the invention disclosed herein (e.g. TEL, Stat3) variants (e.g. derivatives or homologues) of the polypeptides or nucleic acids may also be used in the present invention, provided that they still encode the requisite activity. Generally speaking such variants will be substantially homologous to the ‘wild type’ or other sequence specified herein i.e. will share sequence similarity or identity therewith. Similarity or identity may be at the nucleotde sequence and/or encoded amino acid sequence level, and will preferably, be at least about 50%, 60%, or 70%, or 80%, most preferably at least about 90%, 95%, 96%, 97%, 98% or 99%. Sequence comparisons may be made using FASTA and FASTP (see Pearson &amp; Upman, 1988. Methods in Enzymology 183: 63-98). Parameters are preferably set, using the default matrix, as follows: Gapopen (penalty for the first residue in a gap): −12 for proteins/−16 for DNA; Gapext (penalty for additional residues in a gap): −2 for proteins/−4 for DNA; KTUP word length: 2 for proteins/6 for DNA. Analysis for similarity can also be carried out using hybridisation. One common formula for calculating the stringency conditions required to achieve hybridization between nucleic acid molecules of a specified sequence homology is: Tm=81.5 C+16.6 Log [Na+]+0.41 (% G+C)−0.63 (% formamide)−600/#bp in duplex Molecular Cloning: a Laboratory Manual: 2nd edition, Sambrook et al., 1989, Cold Spring Harbor Laboratory Press).

Exemplary functional equivalents or derivatives (variants) of TEL include molecules where TEL is covalentiy modified by substitution, chemical, enzymatic, or other appropriate means, which may include a moiety other than a naturally occurring amino acid. Derivatives that retain common structural features can be fragments of TEL, in particular fragments maintaining transcription modulatory activity (such as those exemplified in Example 8), Stat3 binding activity, Stat3 modulatory activity or isoform specific characteristics. Preferably, fragments will be between 50 and 350 amino acids in length. Derivatives of TEL also comprise mutants thereof, which may contain amino acid deletions, additions or substitutions, subject to the requirement to maintain at least one feature characteristic of TEL, preferably Stat 3 modulatory activity. Thus, conservative amino acid substitutions may be made substantially without altering the nature of TEL, as may truncations. Additions and substitutions may moreover be made to the fragments of TEL used in the screening methods of the invention, in particular those enhancing TEL activity (e.g., transcription modulatory activity, Stat3 binding activity, Stat3 modulatory activity, HDAC binding activity) or providing some other desirable property.

The screening assays of the invention are not limited to any particular method of determining the presence or activity of TEL The level of TEL can be determined by using an antibody specific for TEL, for example, in an immuno blot. TEL assays are also well known in the art (see for example Van Rompaey et al., 2000, Oncogene, 19, 5244-5250), which describes a method for assaying TEL transcriptional activity by determining Ras-expression or using DNA binding assays. Briefly, TEL will be incubated with a suitable nucleic acid substrate (comprising a TEL binding site), in a buffer allowing binding of TEL to the DNA. Direct binding can be measured using mobility shift assays or by detecting an effect on transcription of a target gene, such as ras or a target bioengineered gene, such as GFP or other detectable marker, as is described in more detail below. Alternatively, antibodies specific for the products of TEL transcription modulatory activity can be used to detect activity. As will be apparent to those of ordinary skill in the art, the assays are easily amenable to high through-put technologies using robotics and automated processes. TEL activity can also be assayed by detecting downstream targets of the protein. For example, TEL is known to affect transcription and translation of a number of specific targets.

A compound-induced modulation of TEL activity or levels means that there is a change in TEL activity (or expression) in the presence of the compound relative to when the compound is absent. In particular a compound induced inhibition of TEL (or its activity) is reflected by a decrease in TEL activity relative to when the compound is absent. Conversely, a compound induced activation of TEL (or its activity) is reflected by an increase in TEL activity in the presence of the compound relative to when the compound is absent.

The modulation can therefore be inhibition of TEL/Etv6 activity, in which case the agent is effective in enhancing cytokine-induced inhibition of cell proliferation. Alternatively, the modulation can be activation of TEL/Etv6 activity, in which case the agent is effective in inhibiting proliferation of cells expressing Stat 3, in particular phosphorylated (activated) Stat3. In the latter case, the cell proliferation can be independent of ras activity. Cancer cells “expressing Stat 3” is meant to encompass cancers overexpressing or having constitutively active Stat3, leading to inappropriate Stat3 signalling.

The methods are particularly useful in identifying agents effective in inhibiting cell proliferation of a melanoma or carcinoma.

Activators and inhibitors are referred to collectively herein as modulators and preferably influence TEL activity directly. Assays carried out using reconstituted components can be easily designed to achieve direct TEL modulation (e.g., specific inhibition of TEL activity).

In accordance with a further aspect of the invention, a method is provided for screening an agent effective in STAT3 dependent disorders, such as cell proliferation, by identifying compounds that modulate the binding of TEL to a binding partner, in particular Stat3. In this aspect of the invention, the method comprises the steps of (i) incubating a TEL/Etv6 polypeptide selected from: TEL/Etv6, a variant or fragment thereof with a binding partner in the presence of a test compound; and (ii) determining whether the presence of a test compound modulates the interaction between the TEL/Etv6 polypeptide and the binding partner relative to when the test compound is absent. As exemplified below in Example 9, the binding partner may be Stat3, a variant or fragment thereof.

Methods for assessing the interaction between a polypeptide and a binding partner may be any of the methods known to those skilled in the art, such as those disclosed here. Any of these methods can be used to assess whether a test compound modulates (e.g., inhibits) the interaction between a polypeptide (in this case TEL) and a binding partner.

In one embodiment, assays are those based upon TEL and its interaction with Stat3, in particular phosphorylated (activated) Stat3. The present inventors have detected TEL interacting with non-phosphorylated Stat3 (in particular, in the cytoplasm of the cell), as well as with phosphorylated Stat3. TEL is generally known as a nuclear protein, and phosphorylated Stat3 shuttles into the nucleus, where it interacts with TEL and modulates transcription.

Determining whether the test compound inhibits the interaction between TEL and a Stat3 polypeptide may comprise determining whether the physical association between the TEL polypeptide and the Stat3 polypeptide is inhibited. For example, the physical association between TEL and a binding partner thereof may be studied by labelling one with a detectable label and bringing it into contact with the other, which has been immobilised on a solid support. Suitable detectable labels include 35S-methionine, which may be incorporated into recombinantly produced TEL and/or the binding partner thereof. The recombinantly produced TEL and/or binding partner may also be expressed as a fusion protein containing an epitope, which can be labelled with an antibody. Alternatively, double-labelling may be used as is well known in the art, for example, using a radioactive label and a scintillant.

Generally, a protein that is immobilised on a solid support may be immobilised using an antibody against that protein bound to a solid support or via other technologies which are known per se. A preferred in vitro interaction may utilise a fusion protein including a tag, such as glutathione-S-transferase (GST) or His6. The tag may be immobilised by affinity interaction, for example on glutathione agarose beads or Ni-matrices, respectively.

In an in vitro assay format of the type described above the putative inhibitor compound can be assayed by determining its ability to modulate the amount of labelled TEL or binding partner, which binds to the Immobilised binding partner, e.g., GST-binding partner or GST-TEL as the case may be. This may be determined by fractionating the glutathione-agarose beads by SDS-polyacrylamide gel electrophoresis. Alternatively, the beads may be rinsed to remove unbound protein and the amount of protein that has bound can be determined by counting the amount of label present in, for example, a suitable scintillation counter.

Alternatively an antibody attached to a solid support and directed against one of TEL or the binding partner may be used in place of GST to attach the molecule to the solid support. Antbodies against TEL and its binding partners may be obtained in a variety of ways known as such in the art.

In an alternative mode, one of TEL and its binding partner may be labelled with a fluorescent donor moiety and the other labelled with an acceptor that is capable of reducing the emission from the donor. This allows an assay according to the invention to be conducted by fluorescence resonance energy transfer (FRET). In this mode, the fluorescence signal of the donor will be altered when TEL and its binding partner interact. The presence of a candidate modulator compound that modulates the interaction will increase the amount of unaltered fluorescence signal of the donor.

FRET is a technique known per se in the art and thus the precise donor and acceptor molecules and the means by which they are linked to TEL and its binding partner may be accomplished by reference to the literature.

Suitable fluorescent donor moieties are those capable of transferring fluorogenic energy to another fluorogenic molecule or part of a compound and include, but are not limited to, coumarins and related dyes such as fluoresceins, rhodols and rhodamines, resorufins, cyanine dyes, bimanes, acridines, isoindoles, dansyl dyes, aminophthalic hydrazines such as luminol and isoluminol derivatives, aminophthalimides, aminonaphthalimides, aminobenzofurans, aminoquinolines, dicyanohydroquinones, and europium and terbium complexes and related compounds.

Suitable acceptors include, but are not limited to, coumarins and related fluorophores, xanthenes such as fluoresceins, rhodols and rhodamines, resorufins, cyanines, difluoroboradiazaindacenes, and phthalocyanines.

A preferred donor is fluorescein and preferred acceptors include rhodamine and carbocyanine. The isothiocyanate derivatives of these fluorescein and rhodamine moieties, available from Aldrich Chemical Company Ltd, Gillingham, Dorset, UK, may be used to label TEL and its binding partner. For attachment of carbocyanine, see for example Guo et al, J. Biol. Chem., 270; 27562-8, 1995.

In accordance with a further aspect of the invention, a method is provided for screening an agent effective in STAT3 dependent disorders, such as cell proliferation, by identifying compounds that modulate expression of a TEL gene or a gene expressed under the control of TEL regulatory sequences.

The methods comprise contacting transcriptionally active cellular components, preferably in a cell, with a nucleic acid encoding a TEL gene operably linked to a promoter sequence or a TEL promoter sequence (or other TEL regulatory regions allowing expression of a reporter gene) operably linked to a reporter gene in the presence of at least one compound; and detecting an effect of the compound on expression of the coding region, be it TEL expression or reporter gene expression. A decrease or an increase in TEL expression or promoter activity is indicative of an agent effective in inhibiting cell proliferation as described above. Such assays can be cell-based assays, where the transcriptonally active cellular components and nucleic acid are present in a cell, although in vitro transcription assays are also well known in the art.

The reporter gene encodes any molecule capable of providing a detectable change. Such reporter molecules include fluorescent moieties (e.g., fluorescent proteins, such as, cyan fluorescent protein, CFP; yellow fluorescent protein, YFP; blue fluorescent protein, BFP; or green fluorescent protein, GFP; all available commercially, Clontech Living Colors User Manual, antigens, reporter enzymes and the like. Reporter enzymes include, but are not limited to, the following: beta-galactosidase, glucosidases, chloramphenicol acetyltransferase (CAT), glucoronidases, luciferase, peroxidases, phosphatases, oxidoreductases, dehydrogenases, transferases, isomerases, kinases, reductases, deaminases, catalases and urease. In selecting a reporter molecule to be used in the presently claimed method, the reporter molecule itself should not be inactivated by any putative agent or other component present in the screening assay, including inactivation by any protease activity present in the assay mixture. The selection of an appropriate reporter molecule will be readily apparent to those skilled in the art.

The nucleic acid will typically be provided in a vector allowing replication in one or more selected host cells, as is well known for a variety of bacteria, yeast, and mammalian cells. For example, various viral origins (SV40, polyoma, adenovirus, VSV or BPV) are useful for cloning vectors in mammalian cells. The vector may, for example, be in the form of a plasmid, cosmid, viral particle, phage, or any other suitable vector or construct which can be taken up by a cell and used to express the sequence of interest or reporter gene. The constructs may be expressed transiently or as stable episomes, or integrated into the genome of the host cell.

Expression vectors usually contain a promoter operably linked to the protein-encoding nucleic acid sequence of interest, so as to direct mRNA synthesis. Promoters recognized by a variety of potential host cells are well known, as are TEL regulatory sequences and promoter sequences. “Operably linked” means joined as part of the same nucleic acid molecule, suitably positioned and oriented for transcription to be initiated from the promoter. DNA operably linked to a promoter is “under transcriptional control” of the promoter. Transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g. the actin promoter or an immunoglobulin promoter, and from heat-shock promoters, provided such promoters are compatible with the host cell systems. Expression vectors of the invention may also contain one or more selection genes, such as genes conferring resistance to antibiotics or other toxins.

The methods of the invention may therefore further include introducing the nucleic acid into a host cell. The introduction, which may (particularly for in vitro introduction) be generally referred to without limitation as “transformation”, may employ any available technique. For eukaryotic cells, suitable techniques may include calcium phosphate transfection, DEAE-Dextran, electroporation, liposome-mediated transfection and transduction using retrovirus or other virus, e.g. vaccini, as is well known in the art. See, for example, Keown et al., Methods in Enzymology, 185:527 537 (1990) and Mansour et al., Nature 336:348-352 (1988).

Host cells transfected or transformed with expression or cloning vectors described herein may be cultured in conventional nutrient media. The culture conditions, such as media, temperature, pH and the like, can be selected by the skilled artisan without undue experimentation. In general, principles, protocols, and practical techniques for maximizing the productivity of cell cultures can be found in “Mammalian Cell Biotechnology: a Practical Approach”, M. Butler, ed. JRL Press, (1991) and Sambrook et al, supra.

Thus, assays of the invention may also be performed in vivo. Such an assay may be performed in any suitable host cell, e.g. a bacterial, yeast, insect or mammalian host cell. Yeast and mammalian host cells are particularly suitable.

In one embodiment, constructs capable of expressing a TEL polypeptide and its binding partner (e.g., Stat3) and a reporter gene construct may be introduced into the cells and the modulation of the reporter gene in response to the presence of a test compound evaluated.

In vivo assays may also take the form of two-hybrid assays. Two-hybrid assays may be in accordance with those disclosed by Fields and Song, 1989, Nature 340; 245-246. In such an assay the DNA binding domain (DBD) and the transcriptional activation domain (TAD) of the yeast GAL4 transcription factor are fused to the respective. interacting molecules under evaluation. A functional GAL4 transcription factor is restored only when two molecules of interest interact. Thus, interaction of the molecules may be measured by the use of a reporter gene operably linked to a GAL4 DNA binding site that is capable of activating transcription of said reporter gene. Other transcriptional activator domains may be used in place of the GAL4 TAD, for example the viral VP16 activation domain.

In general, TEL and its binding partner can be expressed as fusion proteins, one being a fusion protein comprising a DNA binding domain (DBD), such as the yeast GAL4 binding domain, and the other being a fusion protein comprising an activation domain, such as that from GAL4 or VP16. In such a case the host cell (which again may be bacterial, yeast, insect or mammalian, particularly yeast or mammalian) will carry a reporter gene construct with a promoter comprising a DNA binding elements compatible with the DBD.

TEL and its binding partner and the reporter gene, may be introduced into the cell and expressed transiently or stably.

TEL and/or its binding partner may then be contacted with a test compound, and inhibition of binding between TEL and its binding partner can be observed as a reduction of reporter gene expression.

Inhibitors identified in this screen may for example be used to modulate Stat3-dependent cell proliferation, as described above. They may be formulated as medicaments as described hereinafter.

In some embodiments, it is preferred that TEL and/or its binding partner (in particular Stat3) is in its activated form. For example, for in vitro methods, activated TEL or its binding partner may be provided by immunoprecipitation or affinity purification from an activated cell-sample. The cell may be a cell that has been stimulated with a cytokine. For in vivo methods, it may for example be achieved by performing a method in a cell that expresses Stat3, which may be activated by the provision of a ligand, such as a cytokine. Alternatively, it may be constitutively active or phosphorylated (see Bowman et al., 2000, Oncogene, 19, 2474-2488; Bromberg et al. 1999).

Binding assays may be competitive or non-competitive. All assays (whether binding assays or activity assays) will typically run with suitable controls routine to those of skill in the art or alternatively, values determined using the assays compared to standard values previously determined with controls.

The method may optionally further comprise a functional assay to confirm whether the modulator of TEL actvity/binding identified as above is a modulator of Stat3-dependent cell proliferation.

In one embodiment, the method comprises the step of contacting a cell with the modulator of TEL activity/binding, and confirming that the modulator is capable of modulating Stat3-dependent cell proliferation. For example, the modulation may be inhibition of cytokine-sensitive cell proliferation (e.g., using A375 cells as exemplified below cultured in the presence of a cytokine) or inhibition of Stat3 overexpressing cancers (e.g., see Table I and II of Bowman et al., 2000).

The invention further provides methods of identifying an agent effective in modulating Stat3-dependent cell proliferation comprising administering a compound to a non-human animal model dependent on TEL activity and determining whether cell proliferation is affected relative to when the compound is absent. The non-human animals will typically be laboratory mammals such as mice or rats and various doses can be administered orally mixed with feed or by any other appropriate means, which may be chosen dependent on the properties of the compound, such as stability and targeted delivery. The TEL activity may be from a gene expressed by the laboratory animal but from a different species, for example, the method may use a mouse comprising a human TEL gene, which replaces the mouse TEL gene, which will be particularly useful to determine the effects of agents on human TEL without using human subjects.

The screening systems are preferably used to screen compounds that may be present in small molecule libraries, peptide libraries, phage display libraries or natural product libraries. Compounds may be inorganic or organic, for example, an antibiotic or antibody. For ease of administration, the compound is preferably a small molecule, which might bind to the DNA binding site or other transcription regulatory site, Stat3 binding site or indeed to Stat3.

In order to potentially improve TEL modulators, isolated TEL can be used to establish secondary and tertiary structure of the whole protein or at least of the areas responsible for TEL activity. Conventional methods for the identification of the 3-dimensional structure are, for example, X-ray studies or NMR studies. The data obtained with these or comparable methods may be used directly or indirectly for the identification or improvement of modulators of TEL, such as to provide specificity. A commonly used method in this respect is, for example, computer aided drug design or molecular modelling.

Kits useful for screening such compounds may also be prepared in accordance with the invention, and will comprise essentially TEL or a fragment thereof useful for screening, and instructions. Typically the TEL polypeptide will be provided together with means for detecting TEL activity and at least one compound (putative agent) or agents with known modulatory activity, e.g., siRNA, an antibody or antibody fragment. TEL for use in kits according to the invention may be provided in the form of a protein, for example in solution, suspension or lyophilised, or in the form of a nuceic add sequence permitting the production of TEL or a fragment thereof in an expression system, optionally in situ.

The invention also relates to methods and materials for modulating TEL activity, in particular in Stat3-overexpressing cancers or in cytokine-sensitive cancers.

Thus, in one aspect, there is provided a method of reducing TEL activity in a cell for this purpose e.g. by gene silencing. Polynucleotides, in particular antisense RNA or ribozymes against TEL may be used to inhibit TEL activity and may also be inserted into the vectors described above in order to be produced recombinantly or otherwise may be produced synthetically.

An alternative to anti-sense is to use a copy of all or part of the target gene inserted in sense, that is the same, orientation as the target gene, to achieve reduction in expression of the target gene. dsRNA mediated silencing is gene specific and is often termed RNA interference (RNAi) (See Fire (1999) Trends Genet. 15: 358-363, Sharp (2001) Genes Dev. 15: 485-490, Hammond et al. (2001) Nature Rev. Genes 2: 1110-1119 and Tuschl (2001) Chem. Biochem. 2: 239-245). RNA interference is a two step process. First, dsRNA is cleaved within the cell to yield short interfering RNAs (siRNAs) of about 21-23 nt length with 5′ terminal phosphate and 3′ short overhangs (−2 nt). The siRNAs target the corresponding mRNA sequence specifically for destruction (Zamore P. D. Nature Structural Biology, 8, 9, 746-750, (2001)

Thus, double stranded RNA comprising a TEL-encoding sequence, which may for example be a “long” double stranded RNA (which will be processed to siRNA, e.g., using Dicer in vitro) can be used to inhibit Tel activity (and thereby modulate Stat3 activity). These RNA products may alternatively be synthesised in vitro, e.g., by conventional chemical synthesis methods.

RNAi may be also be efficiently induced using chemically synthesized siRNA duplexes of the same structure with 3′-overhang ends (Zamore P D et al Cell, 101, 25-33, (2000)). Synthetic siRNA duplexes have been shown to specifically suppress expression of endogenous and heterologous genes in a wide range of mammalian cell lines (Elbashir S M. et al. Nature, 411, 494-498, (2001)).

Thus siRNA duplexes containing between 20 and 25 bps, more preferably between 21 and 23 bps, of the TEL sequence form one aspect of the invention e.g. as produced synthetically, optionally in protected form to prevent degradation.

Alternatively siRNA may be produced from a vector, in vitro (for recovery and use) or in vivo. Accordingly, the vector may comprise a nucleic acid sequence encoding TEL (or a variant or fragment thereof), suitable for introducing an siRNA into the cell in any of the ways known in the art, for example, as described in any of references cited herein, which references are specifically incorporated herein by reference.

In one embodiment, the vector may comprise a nucleic acid sequence according to the invention in both the sense and antisense orientation, such that when expressed as RNA the sense and antisense sections will associate to form a double stranded RNA. This may for example be a long double stranded RNA (e.g., more than 23 nts), which may be processed by Dicer to produce siRNAs (see for example Myers (2003) Nature Biotechnology 21:324-328).

Alternatively, the double stranded RNA may directly encode the sequences which form the siRNA duplex, as described above. The sense and antisense sequences may alternatively be provided on different vectors.

These vectors and RNA products may be useful for example to inhibit de novo production of the TEL polypeptide in a cell. Thus, a still further aspect of the present invention provides a method, which includes introducing the nucleic acid (e.g. any of the vectors discussed above) into a host cell.

In the light of the disclosure herein it can be seen that modulators of TEL activity include antibodies that bind it specifically and inhibit its interactions with Stat3, thereby having utility in inhibiting Stat3-dependent cell proliferation. TEL antibodies are specific in the sense of being able to distinguish between the polypeptide it is able to bind and other polypeptides of the same species for which it has no or substantially no binding affinity (e.g. a binding affinity of at least about 1000× worse). Specific antibodies bind an epitope on the molecule, which is either not present or is not accessible on other molecules.

Preferred antibodies according to the invention are isolated, in the sense of being free from contaminants such as antibodies able to bind other polypeptides and/or free of serum components. Monoclonal antibodies are preferred for some purposes. Antibodies may be obtained using standard techniques in the art. Methods of producing antibodies include immunising a mammal (e.g. mouse, rat, rabbit) with a polypeptide of the invention. Antibodies may be obtained from immunised animals using any of a variety of techniques known in the art, and screened, preferably using binding of antibody to antigen of interest For instance, Western blotting techniques or immunoprecipitation may be used (Armitage et al, Nature, 357:80-82, 1992).

As an alternative or supplement to Immunising a mammal with a peptide, an antibody specific for a protein may be obtained from a recombinantly produced library of expressed immunoglobulin variable domains, e.g. using lambda bacteriophage or filamentous bacteriophage which display functional immunoglobulin binding domains on their surfaces; for instance see WO92/01047.

Antibodies may be modified in a number of ways and includes antibody fragments, derivatives, functional equivalents and homologues of antibodies, including synthetic molecules and molecules whose shape mimics that of an antibody enabling it to bind an antigen or epitope. Example antibody fragments, capable of binding an antigen or other binding partner are the Fab fragment consisting of the VL, VH, Cl and CH1 domains; the Fd fragment consisting of the VH and CH1 domains; the Fv fragment consisting of the VL and VH domains of a single arm of an antibody; the dAb fragment which consists of a VH domain; isolated CDR regions and F(ab′)2 fragments, a bivalent fragment including two Fab fragments linked by a disulphide bridge at the hinge region. Single chain Fv fragments are also included.

Humanized antibodies in which CDRs from a non-human source are grafted onto human framework regions, typically with the alteration of some of the framework amino acid residues, to provide antibodies which are less immunogenic than the parent non-human antibodies, are also included.

A hybridoma producing a monoclonal antibody according to the present invention may be subject to genetic mutation or other changes. It will further be understood by those skilled in the art that a monodonal antibody can be subjected to the techniques of recombinant DNA technology to produce other antibodies or chimeric molecules which retain the specificity of the original antibody. Such techniques may involve introducing DNA encoding the immunoglobulin variable region, or the complementarity determining regions (CDRs), of an antibody to the constant regions, or constant regions plus framework regions, of a different immunoglobulin. See, for instance, EP-A-184187, GB-A-2188638 or EP-A-0239400. Cloning and expression of chimeric antibodies are described in EP-A-0120694 and EP-A-0125023.

On the other hand, low molecular weight compounds are primarily indicated as therapeutic agents, which are preferably produced by chemical synthesis according to established procedures. These compounds may modulate posttranslational modification of TEL, such as sumolation or phosphorylation or expression of TEL. Sumolation leads to nuclear export of TEL and hence deactivation. Phosphorylation of TEL might be important for TEL activation. The low molecular weight compounds and organic compounds (including antibodies) in general may be useful as agents for use in modulating Stat3-dependent cell proliferation and preferably act selectively on TEL.

Compounds according to the invention may be identified by screening using the techniques described hereinbefore, and prepared by extraction from natural or genetically modified sources according to established procedures, or by synthesis, especially in the case of low molecular weight chemical compounds. Proteinaceous compounds may be prepared by expression in recombinant expression systems, for example a baculovirus system, or in a bacterial system. Proteinaceous compounds are mainly useful for research into the function of signalling pathways, although they may have a therapeutic application, such as humanized inhibitory antibodies directed against TEL. Alternatively, nucleic acids, such as siRNA can be administered to inhibit TEL activity. SiRNA technology can be routinely applied based on sequences specific for TEL, such as those described below in the Examples. Targeted expression of siRNAs can be achieved using tissue-specific promoters.

Also provided by the invention are methods of inhibiting Stat3 expressing cancer cell proliferation, in particular melanomas or carcinomas, comprising contacting a cancer cell expressing Stat3, in particular phosphorylated Stat3, with an effective amount of an activator of TEL in an amount sufficient to inhibit Stat3 activity. Preferably, the cell proliferation is independent of ras.

In a further embodiment, the invention provides a method of inhibiting cytokine sensitive cancer cell growth, comprising contacting a cytokine-sensitive cancer cell with an effective amount of a TEL inhibitor in an amount sufficient to enhance Stat3 activity. The TEL inhibitor can be an RNAI or an inhibitory antibody (or fragment thereof), for example.

Thus, also provided by the invention are compounds that directly modulate TEL for use in inhibiting STAT3-dependent cell proliferation. TEL modulators (e.g., inhibitors) may be formulated according to conventional methodology, depending on the exact nature of the modulator, and will typically comprise the modulator or a precursor thereof in association with a biologically acceptable carrier. In considering various therapies, it is understood that such therapies may be targeted to tissues demonstrated to express TEL and/or Stat3, in particular phosphorylated Stat3.

Delivery of the modulator to the affected cells and tissues can be accomplished using appropriate packaging or administration systems. For example, the modulator may be formulated for therapeutic use with agents acceptable for pharmaceutical administration and delivered to the subject by acceptable routes to produce a desired physiological effect. An effective amount is that amount that produces the desired physiological effect, such as, a reduction in cell proliferation.

In a further aspect of the invention, the invention also provides a TEL modulator for the manufacture of a medicament for the treatment or prophylactic treatment of Stat3-dependent cell proliferation.

Thus the relevant methods may include the further step of formulating a selected modulator (e.g., inhibitor as a medicament for a disease e.g. in which it is desired to control Stat3-dependent cell proliferation e.g. the treatment of tumors, including head and neck cancers, HTLV-1 dependent leukaemia, acute myeloid leukaemia, chronic lymphocyte leukemia, large granular lymphocyte leukaemia, lymphomas, renal cell carcinoma, prostate carcinoma, melanoma, pancreatic adenocarcinoma and ovarian carcinoma. Such inhibitors and medicaments for use in the treatment of these diseases, and methods of treatment comprising their use form further aspects of the invention.

The term treatment as used herein is intended to include prophylaxis and prevention as well as alleviation the condition or symptoms thereof.

The compositions may include, in addition to the above constituents, pharmaceutically-acceptable excipients, preserving agents, solubilizers, viscosity-increasing substances, stabilising agents, wetting agents, emulsifying agents, sweetening agents, colouring agents, flavouring agents, salts for varying the osmotic pressure, buffers, or coating agents. Such materials should be non-toxic and should not interfere with the efficacy of the active ingredient. The precise nature of the carrier or other material may depend on the route of administration. Examples of techniques and protocols can be found in “Remington's Pharmaceutical Sciences”, 16th edition, Osol, A (ed.), 1980.

Where the composition is formulated into a pharmaceutical composition, the administration thereof can be effected parentally such as orally, nasally (e.g. in the form of nasal sprays) or rectally (e.g. in the form of suppositories). However, the administration can also be effected parentally such as intramuscularly, intravenously, cutaneously, subcutaneously, or intraperitoneally (e.g. in the form of injection solutions).

Thus, for example, where the pharmaceutical composition is in the form of a tablet, it may include a solid carrier such as gelatine or an adjuvant. For the manufacture of tablets, coated tablets, dragees and hard gelatine capsules, the active compounds and their pharmaceutically-acceptable acid addition salts can be processed with pharmaceutically inert, inorganic or organic excipients. Lactose, maize, starch or derivatives thereof, talc, stearic acid or its salts etc. can be used, for example, as such excipients for tablets, dragees and hard gelatine capsules. Suitable excipients for soft gelatine capsules are, for example, vegetable oils, waxes, fats, semi-solid and liquid polyols etc. Where the composition is in the form of a liquid pharmaceutical formulation, it will generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may also be included. Other suitable excipients for the manufacture of solutions and syrups are, for example, water, polyols, saccharose, invert sugar, glucose, trihalose, etc. Suitable excipients for injection solutions are, for example, water, alcohols, polyols, glycerol, vegetable oils, etc. For intravenous, cutaneous or subcutaneous injection, or intracatheter infusion into the brain, the active ingredient will be in the form of a parenterally-acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers and/or other additives may be included, as required.

The invention also provides a method of diagnosing Stat3-dependent cancer, comprising detecting the level of TEL, in the sample and correlating a change in the amount of TEL or its activity in the sample when compared to a normal control value or range of values with a predisposition to Stat3-dependent cancer. The presence of TEL can be easily determined using antibodies or using activity assays as described above. For example, an increase in TEL of at least 20 %, preferably at least 30% when compared to a normal control value or range of values is indicative of a predisposition to a Stat3-dependent cancer (having constitutively active Stat3). The sample may be any biopsy or body fluid.

Although the description above is primarily concerned with TEL/Etv6, it will be apparent to one of ordinary skill in the art in light of the present disclosure that any one of the genes/gene products referred to in Table 4 below can be modulated to inhibit Stat3-dependent cell proliferation. Therefore it would be well within the skill of the art to design screening assays and methods of inhibiting cell proliferation based on the teachings above relating to TEL/Etv6 but using the cell targets referred to in Table 4 instead of TEL/Etv 6 (e.g., STAT inhibitor 3).

These genes can be appointed to various functional groups, such as transcriptional regulators mediating a Stat3-dependent response; receptors (e.g., the cytokine receptor, OSM receptor and cytokine receptor-like factor-1, which belong to the IL-6 type receptor family); intracellular signaling molecules; adhesion proteins (such as osteopontin) and genes involved in various aspect of cellular metabolism. For example, the expression of SOCS3, an inhibitor of Jak/Stat signaling, C/EBPdelta, JunB and alpha-antichymotrypsin (serpin A3) were significantly increased upon 4HT treatment. Similarly, death-associated protein kinase 1 (DAPK1) and the serine protease inhibitor serpin B3 also increased upon 4HT treatment. Thus, for example, DAPK1 activity can be modulated to inhibit cell proliferation. Where it is desired to inhibit Stat3 activity (such as in phosphorylated Stat3-expressing cancers or otherwise constitutively active Stat3 expressing cancers), this may be achieved by inhibiting DAPK activity (or one of the other gene products listed in Table 4, such as secreted phosphoprotein 1). Similarly, where it is desired to enhance Stat3 activity (such as in cytokine sensitive cancers), the protease inhibitor serpin B3 may be administered.

The invention is further described, for the purposes of illustration only, in the following examples.

### EXAMPLES

Methods of molecular genetics, protein and peptide biochemistry and immunology referred to but not explicitly described in this disclosure and examples are reported in the scientific literature and are well known to those skilled in the art. For example, standard methods in genetic engineering are carried out essentially as described in Sambrook et al., Molecular Cloning: A laboratory manual, 2nd Ed., Cold Spring Harbor Laboratory Press, Cold Spring Harbor N.Y., 1989.

### Example 1

### 4-Hydroxytamoxifen Induces Stat3ER DNA Binding and Transcriptional Activity

Inhibition of breast carcinoma and melanoma cell proliferation by IL-6-type cytokines is dependent on Stat3. An inducible Stat3 construct was designed to allow the investigation of the role of Stat3 in the inhibition of cell proliferation, independently of other IL-6-induced signaling pathways. The inducible Stat3 construct comprises the entire coding sequence of Stat3 (Accession No. NM_{—}139276) fused to a mutated estrogen receptor ligand binding domain (ER-LBD) and is referred to herein as Stat3-ER. Addition of 4-hydroxytamoxifen (4HT), an ER-LBD ligand, triggers Stat3-ER dimerization, translocation to the nucleus and activation of Stat3-dependent transcriptional activity.

Briefly, a plasmid comprising the mutated ligand-binding domain of the murine estrogen receptor (T Littlewood, Nucleic Acids Res, 1995, 23, 1686-90) ERTM was cloned into pcDNA 3.1 (commercially available from Invitrogen) as a BamH1-EcoR1 fragment. Stat3 sequence with an additional BamH1 site was obtained by PCR amplification of pRC/CMV Stat3 (J. Bromberg, Proc Natl Acad Sci USA., 2001, 98, 1543-8) and cloned into pcDNA3.1-ERTM.

IL-6 and oncostatin M (OSM) inhibit A375 melanoma cell proliferation. The Stat3-ER construct was transfected into the A375 melanoma cell line and several clones expressing high levels of Stat3-ER were selected. A375 melanoma cells were maintained in RPMI supplemented with 5% FCS (Life Technologies, Inc., Grand Island, N.Y.). Plasmids were introduced into cells using Effectene transfection reagent (Qiagen, Hilden, Germany) according to the manufacturer's protocol. Cells were selected in 1 mg/ml G418, and several clones were picked, expanded in the presence of G418, and analyzed for STAT3-ER expression. Similar results were obtained with two independent STAT3-ER clones (AER18 and AER2).

DNA binding assays were carried out using cells stimulated with 4HT (1 microM) for 0, 0.3, 1, 2, 4 and 24 hours. Nuclear extracts were prepared as described (Andrew and Faller, 1991). Stat3 Trans Cruz Oligonucleotide Agarose Conjugates (20 microliters; Santa Cruz Biotechnology, 5′-GAT CCT TCT GGG AAT TCC TAG ATC-3′; SEQ ID NO:1) were added to 80 microgrammes of nuclear extract in binding buffer (20 mM HEPES, 0.5 mM EDTA, 1 mM DTT, 2 microgrammes/ml poly dI-dC) for 2 hours at 4° C. with rotation. Samples were centrifuged and the pellet was washed two more times with binding buffer. Proteins were eluted from beads by boiling in 25 microliters of 2× loading buffer (1% DTT, 4% SDS, 20% Glycerol, 0.02% Bromophenol Blue, 0.25 M TrisHCl 1M 6.8 pH) and SDS-PAGE was performed.

While Stat3ER-DNA binding was detected as early as 20 min after stimulation, binding increased strongly after two hours of simulation to reach a maximum measured level after 24 hrs of 4HT stimulation. Thus, addition of 4HT to Stat3-ER-expressing A375 cells induced nuclear translocation and DNA-binding to a Stat3-specific oligonucleotide probe. The increase in DNA binding was accompanied by an increase in Stat3-ER expression, probably due to ligand-dependent stabilization of the Stat3-ER fusion protein.

Luciferase assays are used to verify that the Stat3-ER construct is transcriptionally active. For this purpose, a luciferase reporter gene driven by the acute-phase response element (APRE) of the alpha 2-macroglobulin gene, a target for Stat1 and Stat3, was used.

Cells (2×10⁶ cells/6 well dish) were transfected with a reporter plasmid containing four copies of acute phase responsible element (APRE) upstream of the minimal junB promoter linked to the luciferase gene (T Hirano, EMBO Journal, 1996, 15, 3651-3658), together with the internal control plasmid pRL-SV40 from Promega (Madison, Wis.). Twenty-four hours after the transfection, cells were either left unstimulated or stimulated with OSM (PeproTechEC, London, United Kingdom), 4-hydroxytamoxifen (4HT; Sigma, St. Louis, Mo.) or a combination of 4HT/OSM for 24 hours. The preparation of cell extracts and measurement of luciferase activities were carried out using the Dual-Luciferase reporter assay system from Promega (Madison, Wis.) in an Autolumat LB 953 (Berthold Technologies, Wildbad, Germany). Changes in firefly luciferase activity were expressed relative to Renilla luciferase activity in the same sample.

In A375 cells, oncostatin M (OSM) was the most potent activator of Stat3 transcriptional activity among the IL-6 family members tested, which included interleukin 6 (IL-6) and leukemia inhibitory factor (LIF). In Stat3-ER expressing A375 cells, 4HT (1 microM) stimulates transcription from the APRE, more efficiently than 100 ng/ml OSM. The results are presented in Table 1 below:

TABLE 1

| | Relative Luciferase |
|---|---|
| Sample | Activity |
| Control | 1 |
| OSM (1 ng/ml) | 3 |
| OSM (10 ng/ml) | 9 |
| OSM (100 ng/ml) | 18 |
| 4HT (1 microM) | 29 |
| OSM (1 ng/ml) + 4HT (1 microM) | 47 |
| OSM (10 ng/ml) + 4HT (1 microM) | 2853 |
| OSM (100 ng/ml) + 4HT (1 microM) | 7704 |

We have determined 1 microM to be the optimal concentration of 4HT in this assay under these conditions. In the APRE-driven reporter assay, the combination of 4HT and OSM dramatically increased luciferase activity, compared to the activity induced by 4HT or OSM alone. At the highest dose of OSM (100 ng/ml), transcription induced by combination with 4HT was more than 100-fold stronger than the transcriptional activity of 4HT or OSM used individually. IL-6 shows the same cooperative effect with 4HT. Thus, in STAT3-ER-expressing A375 cells, 4HT and gp130 signaling synergize to increase Stat3 transcriptional activity. This Example also demonstrates that Stat3-ER can be used effectively to establish the effects of Stat3 on cell proliferation.

### Example 2

### STA3 DNA-Binding is Dependent on Tyr 705 Phosphorylation

STAT3-ER-expressing A375 cells were treated with 4HT for 24 hrs essentially as described in Example 1 and then OSM was added for 15 minutes, 30 minutes, 1 hr, 2 hr, 4 hr or 24 hr. DNA binding of Stat3-ER and endogenous Stat3 was monitored by performing Western analysis on the oligonucleotide-bound proteins. Stat3 DNA binding could be detected 15 min after addition of OSM and decreased after 1 hr of treatment. Due to pretreatment with 4HT, Stat3-ER was DNA bound before OSM addition. In the presence of OSM, Stat3-ER DNA binding was enhanced and, in contrast to endogenous Stat3 remained bound for the entire course of the experiment. The DNA binding kinetics of Stat3 and Stat3-ER correlated essentially with Tyr705 phosphorylation levels. These results demonstrate that OSM-induced gp130 activation induces phosphorylation of both Stat3 and Stat3-ER; while Stat3 gets rapidly de-phosphorylated, Stat3-ER remains phosphorylated and therefore DNA-bound for prolonged periods. This result very likely explains the synergy between 4HT and OSM in stimulating Stat3-dependent transcriptional activity and Stat3-induced inhibition of cell proliferation.

### Example 3

### Stat3 Activity Inhibits A375 Cell Proliferation

In order to evaluate the role of Stat3 in A375 cell proliferation, A375-Stat3-ER cells were grown in the presence of OSM or 4HT and cell number determined over 3 days. At a concentration of 1 microM, 4HT does not affect proliferation of the parent A375 cells. Cells were grown in 35 mm-dishes and total cell number was determined at 0, 24, 48 and 72 hours after treatment with OSM and/or 4HT in a hemocytometer. The values are provided in Table 2 below.

TABLE 2
Number of Cells(×10⁵)

| Time | Control | OSM | 4HT | OSM + 4HT |
|---|---|---|---|---|
| 0 | 0.8 | 0.8 | 0.8 | 0.8 |
| 24 h | 1.48 | 1.15 | 1.49 | 1.31 |
| 48 h | 4.33 | 2.58 | 3.60 | 2.25 |
| 72 h | 7.83 | 4.35 | 4.44 | 1.50 |

Upon 4HT treatment (1 microM) A375-Stat3ER cell number was reduced by about 40%, which is equivalent to the effect of 100 ng/ml OSM. Thus, Stat3 activity is not only required, but also sufficient to inhibit A375 melanoma cell proliferation. When combined, 4HT and OSM induced a marked decrease in A375Stat3-ER cell number.

To further delineate the role of Stat3 activation in decreasing cell number, cell cycle analysis of A375-Stat3-ER cells was preformed, upon 4HT, OSM or combined 4HT/OSM treatment. In brief, cells were harvested at the indicated times (24, 48 and 72 h) washed three times with ice-cold PBS and resuspended in propidium iodide buffer (1 mM sodium citrate (pH 4.0), 1.5 mM NaCl, 5 mM EDTA, 5 mM EGTA, 0.1% NP40, 4 mg/ml propidium iodide and 80 mg/ml of RNase A). After 30 min incubation in the dark on ice, cell cycle distribution was measured with a Becton Dickinson FACS can flow cytometer. The results are shown in Table 3 below:

TABLE 3

| Treatment/Time | G1 | S | G2/M | Apoptotic cells |
|---|---|---|---|---|
| 4HT 24 h | +1% | −3% | +2% | <1% |
| 4HT 48 h | +5% | +1% | −6% | <1% |
| 4HT 72 h | −1% | 0% | +1% | <1% |
| 4HT + OSM 24 h | +24% | −21% | −3% | <1% |
| 4HT + OSM 48 h | +17% | −7% | −17% | 8% |
| 4HT + OSM 72 h | −9% | −5% | −7% | 21% |

4HT only marginally affected cell cycle profile, inducing a transient 5% increase in the percentage of cells in the G1 phase of the cell cycle after 48 h of treatment. Cell cycle was normal again after 3-days' treatment, a time when cell number is decreased by about 40%. likewise, OSM alone had little effect on the A375 cell cycle profile. In contrast, upon combined addition of 4HT and OSM, cells underwent a rapid (24 hrs) block in the G1 phase of the cell cycle. At 48 hrs, cells accumulated in G1 and started to die from apoptosis as indicated by the appearance of a sub-G1 peak and PARP cleavage. The percentage of apoptotic cells reached 20% after 3 days of stimulation. Thus, the reduction in cell number induced by the combined action of 4HT and OSM is largely due to STAT3-mediated cell death.

While cell distribution in the different phases of the cell cycle was not affected, the effect of Stat3 activation on cell cycle can be observed upon release of A375 Stat3-ER cells synchronized in G1. Entry of 4HT-treated cells into S phase was delayed relative to control cells. These results indicate that while not affecting cell cycle distribution, Stat3 activity delays progress through the cell cycle.

### Example 4

### Transcriptional Profiling of Stat3 Genes

In order to understand Stat3's ability to affect cell proliferation and cell death, we have examined the profile of genes expressed upon Stat3 activation in A375 cells using oligonucleotide microarrays. Experiments were carried out on two independent clones, expressing similar amounts of Stat3-ER and showing a similar response in DNA binding, Stat3-driven reporter assay and inhibition of cell proliferation. DNA binding experiments and reporter assays indicated that Stat3 binding and transcriptional activity was strongly increased between 2 and 4 hrs after 4HT addition (see Example 1). This was confirmed by microarray experiments.

Briefly, microarray analysis was performed using HG U95A GeneChips™ (Affymetrix, Santa Clara, USA) essentially according to the manufacturer's instructions. Stat3-ER expressing cells were stimulated with 4HT for 4, 24 and 72 hrs before RNA collection. RNA from three independent experiments was pooled and and biotin-labeled cRNA probes were generated from each sample starting from 10 μg of total cellular RNA. The resulting biotinylated cRNAs were hybridized to the Affymetrix U95A oligonucleotide array containing more than 12,000 sequences and signals detected. Chip analysis was performed using the Affymetrix Microarray Suite v5 (target intensity 500 used for chip scaling) and GeneSpring 4.2.1 (Silicon Genetics). Changes in gene expression were assessed by looking for concordant changes between replicates using a signed Wilcoxon rank test (as recommended by Affymetrix). The “change” p-value threshold was <0.003 for an increase in expression and >0.997 for a decrease in expression. After concordance analysis these values become <9×10⁻⁶ and >0.999991 respectively. Any gene whose detection p-value was >0.05 in all experimental conditions was discarded from the analysis as being unreliable data.

Genes that were significantly increased or decreased in both clones relative to untreated cells (p<0.003) were therefore identified, but only gene whose expression was changed more than two-fold were selected for further analysis. Genes that were significantly increased or decreased upon 4HT in non-transfected A375 cells, were excluded, unless the change in Stat3-ER expressing cells were more than double in both clones relative to the change in A375 cells.

Several known Stat3 target genes were induced in our system, validating our experimental approach (Table 4).

TABLE 4

| 4HT | | | OSM | | | |
|---|---|---|---|---|---|---|
| Clone1 | Clone2 | Avge | Clone1 | Clone2 | Avge | Description |
| | | | | | | Nuclear Proteins |
| 3.6 | 6.3 | 4.9 | 1.4 | 1.0 | 1.2 | v-jun avian sarcoma virus 17 oncogene |
| | | | | | | homologue |
| 5.6 | 3.7 | 4.6 | 3.9 | 1.6 | 2.7 | CCAAT/enhancer binding protein |
| | | | | | | (C/EBP), delta |
| 5.6 | 3.1 | 4.3 | 1.8 | 1.1 | 1.5 | v-jun avian sarcoma virus 17 oncogene |
| | | | | | | homologue |
| 3.9 | 4.5 | 4.2 | 3.9 | 4.0 | 3.9 | Ets variant gene 6 |
| 3.6 | 4.1 | 3.9 | 2.9 | 4.6 | 3.7 | v-fos FBJ murine osteosarcoma viral |
| | | | | | | gene homologue |
| 3.3 | 2.6 | 3.0 | 2.7 | 2.2 | 2.5 | Jun B proto-oncogene |
| 1.2 | 2.0 | 1.6 | 2.5 | 3.5 | 3.0 | v-maf musculoaponeurotic fibrosarcoma |
| | | | | | | oncogene homologue (avian) |
| | | | | | | Intracellular signalling |
| 9.3 | 11.7 | 10.5 | 4.5 | 10.2 | 7.4 | Prostatic acid phosphatase precursor |
| 9.2 | 10.5 | 9.9 | 11.6 | 9.7 | 10.7 | STAT induced STAT inhibitor 3 |
| 6.9 | 3.7 | 5.3 | 3.7 | 2.1 | 2.9 | Regulator of G protein signalling 16 |
| 5.7 | 4.8 | 5.3 | 3.5 | 6.9 | 5.2 | Death-associated protein kinase 1 |
| 2.5 | 5.4 | 3.9 | 2.6 | 2.1 | 2.4 | Insulin-like growth factor binding protein 3 |
| 2.3 | 3.9 | 3.1 | 2.4 | 1.7 | 2.0 | Insulin-like growth factor binding protein 3 |
| 3.3 | 2.4 | 2.8 | 1.0 | 1.7 | 1.3 | Prostatic acid phosphatase precursor |
| 2.3 | 2.1 | 2.2 | 0.9 | 1.6 | 1.2 | Tyrosine |
| | | | | | | phosphatase/TIGR = HG620-HT620 |
| | | | | | | Growth Factors/cytokines/receptors |
| 18.4 | 2.2 | 10.3 | 6.7 | 0.4 | 3.6 | Killer cell lectin-like receptor subfamily |
| | | | | | | C, member 3 isoform NKG2-E |
| 12.9 | 4.4 | 8.6 | 5.2 | 0.7 | 2.9 | Osteoprotegrin |
| 8.9 | 3.6 | 6.3 | 4.8 | 4.7 | 4.8 | Brain-derived neurotrophic factor |
| 9.5 | 2.7 | 6.1 | 4.3 | 1.0 | 2.7 | Killer cell lectin-like receptor subfamily |
| | | | | | | C, member 2 |
| 2.3 | 7.4 | 4.9 | 0.4 | 0.6 | 0.5 | Interleukin 8 |
| 5.2 | 4.3 | 4.8 | 3.0 | 0.9 | 1.9 | Cytokine receptor-like factor 1 |
| 2.8 | 5.2 | 4.0 | 1.3 | 2.9 | 2.1 | Ephrin A1 precursor |
| 4.3 | 2.4 | 3.3 | 1.9 | 2.1 | 2.0 | Oncostatin M receptor |
| 4.2 | 2.5 | 3.3 | 1.9 | 2.0 | 1.9 | Oncostatin M receptor |
| 2.9 | 2.2 | 2.6 | 1.2 | 0.7 | 0.9 | Interleukin 8 |
| 2.8 | 2.0 | 2.4 | 0.9 | 1.5 | 1.2 | Small inducible cytokine A2 (monocyte |
| | | | | | | chemotactic protein 1) |
| | | | | | | Cell |
| | | | | | | metabolism/transporters/channels |
| 5.2 | 8.3 | 6.7 | 2.8 | 4.6 | 3.7 | Stanniocalcin 1 |
| 5.2 | 5.1 | 5.2 | 4.5 | 3.8 | 4.1 | Aquaporin 3 |
| 5.7 | 3.2 | 4.4 | 9.2 | 6.7 | 8.0 | Aquaporin 3 |
| 3.6 | 3.8 | 3.7 | 2.3 | 3.4 | 2.8 | Cathepsin L |
| 4.0 | 3.2 | 3.6 | 2.6 | 1.7 | 2.2 | Kynureninase (L-kynurenine hydrolase) |
| 4.3 | 2.4 | 3.3 | 1.8 | 1.3 | 1.5 | Kynureninase (L-kynurenine hydrolase) |
| 3.9 | 2.0 | 2.9 | 1.9 | 1.4 | 1.7 | Kynureninase (L-kynurenine hydrolase) |
| 2.9 | 2.3 | 2.6 | 1.9 | 2.6 | 2.3 | Solute carrier family 2 (facilitated |
| | | | | | | glucose transporter), member 3 |
| 2.7 | 2.5 | 2.6 | 2.8 | 1.9 | 2.4 | Nicotinamide N-methyltransferase |
| 1.8 | 1.6 | 1.7 | 2.5 | 2.5 | 2.5 | Glycine dehydrogenase |
| | | | | | | (decarboxylating, glycine |
| | | | | | | decdarboxylase, glycine cleavage |
| | | | | | | system protein P) |
| | | | | | | Cell-cell interaction/cell adhesion |
| 10.8 | 40.1 | 25.5 | 3.8 | 11.3 | 7.5 | Secreted phosphoprotein 1 |
| | | | | | | (osteopontin, bone sialoprotein 1, early |
| | | | | | | T-lymphocyte activation 1) |
| 16.9 | 20.0 | 18.4 | 3.5 | 5.7 | 4.6 | Secreted phosphoprotein 1 |
| | | | | | | (osteopontin, bone sialoprotein 1, early |
| | | | | | | T-lymphocyte activation 1) |
| 7.7 | 2.6 | 5.2 | 1.2 | 1.9 | 1.6 | Fibronectin 1, isoform 1 preproprotein |
| 6.8 | 3.3 | 5.0 | 0.6 | 2.3 | 1.5 | Fibronectin 1, isoform 1 preproprotein |
| 4.3 | 4.6 | 4.5 | 0.9 | 3.1 | 2.0 | Fibronectin |
| 3.1 | 2.5 | 2.8 | 2.2 | 0.6 | 1.4 | Interferon induced transmembrane |
| | | | | | | protein 1 (9-27) |
| 2.8 | 2.1 | 2.4 | 1.7 | 2.1 | 1.9 | Intercellular adhesion molecule 1 |
| | | | | | | precursor |
| 2.3 | 2.2 | 2.2 | 1.7 | 2.4 | 2.1 | Niemann-Pick disease, type C1 |
| | | | | | | Inflammation/pathogen defense |
| 3.8 | 2.0 | 2.9 | 2.5 | 2.2 | 2.3 | I factor (complement) |
| 3.0 | 2.4 | 2.7 | 3.4 | 2.6 | 3.0 | MHC class II transactivator |
| 2.7 | 2.3 | 2.5 | 3.1 | 2.4 | 2.8 | Alpha-1-antichymotrypsin precursor |
| 3.0 | 1.3 | 2.2 | 2.5 | 2.3 | 2.4 | MHC, class II, DQ alpha 1 precursor |
| 1.8 | 2.0 | 1.9 | 2.4 | 2.6 | 2.5 | MHC, class II, DQ beta 1 |
| | | | | | | Other functions |
| 103.0 | 97.5 | 100.2 | 28.8 | 26.7 | 27.8 | Serine (or cysteine) protease inhibitor, |
| | | | | | | clade B (ovalbumin), member 3 |
| 1.6 | 6.7 | 4.2 | 2.3 | 3.8 | 3.0 | KIAA0500 protein |
| 4.7 | 3.4 | 4.0 | 3.0 | 3.0 | 3.0 | Regenerating islet-derived 1 alpha |
| | | | | | | (pancreatic stone protein, pancreatic |
| | | | | | | thread protein) |
| 4.2 | 1.7 | 2.9 | 4.5 | 2.4 | 3.5 | KIAA0303 protein |
| 3.6 | 2.0 | 2.8 | 2.9 | 2.5 | 2.7 | Major vault protein |
| 2.6 | 2.0 | 2.3 | 1.5 | 1.6 | 1.5 | H. sapiens mRNA; cDNA |
| | | | | | | DZFZp586N012 |
| 2.5 | 2.4 | 2.4 | 1.5 | 1.7 | 1.6 | KIAA0161 gene product |
| 2.4 | 2.5 | 2.4 | 1.8 | 1.9 | 1.8 | Ras-induced senescence 1 |
| 2.2 | 2.0 | 2.1 | 2.7 | 3.1 | 2.9 | Absent in melanoma 2 |

For example, SOCS3, an inhibitor of Jak/Stat signaling, which is involved in the negative feedback of Stat signaling by interfering with Jak activity, was rapidly increased upon 4HT treatment (as early as 1 hr after treatment). Similarly, C/EBPdefta, JunB and alpha-antichymotrypsin (serpin A3) were significantly increased upon 4HT treatment. However, other Stat3 target genes were also activated rapidly, and were therefore identified as novel Stat3 target genes. These include the TEL/Etv6 transcription factor, death-associated protein kinase 1 (DAPK1) and the serine protease inhibitor serpin B3.

Globally 4HT-induced Stat3 activation resulted in alteration of the expression of a limited number of genes. Indeed, after 24 hrs of treatment, 154 genes were significantly increased and 23 decreased, but only 39 genes were upregulated more than two-fold (Table 4). The sequences of the genes are well known in the art and are provided in various databases, such as Genbank. The genes can be appointed to various functional groups, such as transcriptional regulators mediating a Stat3-dependent response; receptors (e.g., the cytokine receptor, OSM receptor and cytokine receptor-like factor-1, which belong to the IL-6 type receptor family); intracellular signaling molecules; adhesion proteins (such as osteopontin) and genes involved in various aspect of cellular metabolism.

The results of the microarray screen for 4HT-induced genes in Stat3ER-expressing A375 cells were corroborated using a more physiological Stat3 activator, namely OSM. Because OSM activates multiple pathways besides Stat3 through gp130, such as MAPK, ribosomal S6 kinase or protein kinase C, we expected OSM-induced transcriptional profile to be broader, but to include 4HT targets. In Stat3-ER expressing cells, most 4HT targets were also OSM targets (Table 4). However, OSM induced a more moderate transcriptional response and therefore there were fewer genes increased above the two-fold threshold. Some genes including fibronectin, interleukin-8 and c-Jun appear not to be OSM targets. Conversely, a few genes appeared to be more efficiently induced by OSM than 4HT. Transcriptional activation of these genes, which includes members of the class II major histocompatibility complex, the putative tumor suppressor Aim2 and the proto-oncogene c-Maf may be dependent not only on activation of Stat3, but also on alternative pathways such as the MAPK or the PI3K pathway.

### Example 5

### Role of C/EBPdelta in Stat3-Mediated Inhibition of Cell Proliferation

Small interfering (si) RNAs are used to disrupt the expression of genes identified in Example 3 as playing a role in Stat3-induced inhibition of cell proliferation. This Example follows cell proliferation of Stat3-ER-expressing A375 cells treated with 4HT or a combination of 4HT and OSM, then cell proliferation in the absence or in the presence of siRNA to C/EBPd.

For siRNA transfection, cells were plated in six-well plates one day before transfection at a density of 5×10⁴ cells/well. siRNA duplexes were introduced using the OligofectatAMINE reagent (Life Technologies) according to the manufacturer's protocol with 10 microliters of 20 microM siRNA and 3 microliters of transfection reagent/well. The following 21-mer oligoribonucleotide pairs were used:

| | |
|---|---|
| C/EBPdelta siRNA nt 844-864, | |
| 5′-GCAGCUGCCCAGCCCGCCCdTdT-3′ | (SEQ ID NO:2) |
| and | |
| 5′-GGGCGGGCUGGGCAGCUGdTdT-3′. | (SEQ ID NO:3) |

A LacZ siRNA was used as control. The designed RNA oligonucleotides were ‘blasted’ against the GENBANK/EMBL databank to ensure gene specificity. The RNA oligonucleotides were obtained commercially. Transfection with expression vectors was carried out 1 day after the siRNA transfection using Effectene. When examined by quantitatve PCR, C/EBPdelta mRNA was significantly increased upon 4HT treatment and strongly increased by the OSM/4HT combination, confirming the data obtained by microarray analysis. C/EBPdelta siRNA significantly reduced expression of C/EBPdelta. In the presence of control siRNA, about 40-45% inhibition of cell proliferation was observed. In the presence of C/EBPdelta siRNA, inhibition of A375 cell proliferation was significantly reduced (˜40%, average of three independent experiments, i.e., a reduction to only about 25% inhibition of cell proliferation), indicating that C/EBPdelta is a mediator of Stat3 function.

### Example 6

### TEL is a Negative Regulator of Stat3 Activity

Microarray data indicate that TEL mRNA is increased within 4 hrs of Stat3 activation. TEL is usually expressed as two protein isoforms corresponding to initiation of TEL mRNA translation at ATG codon 1 and 43 (TEL and TEL43). Both isoforms of TEL were significantly increased within 8 hrs of 4HT addition to ASER cells and strongly increased after 24 hrs of treatment. To ascertain if TEL is a natural target of Stat3, we examined its expression in human cancer cell lines in response to OSM or IL-6. Indeed TEL expression was significantly increased following IL-6/OSM treatment of T47D and SKBr-3 breast cancer cells, DU145 prostate carcinoma cells and HepG2 hepatoma-derived cells. However, TEL expression was unchanged in other cell lines, including the prostate cancer cells PC3 and LNCaP and MDA-MB-468 breast cancer cells, even though it was expected that Stat3 is activated in each of these cell lines by IL-6/OSM treatment.

Experiments were performed to investigate the function of TEL in Stat3-mediated inhibition of cell proliferation essentially as described in Example 4 but using TEL siRNA nt 540-560, 5′-CCCUCCCACCAUUGAACUGdTdT-3′ (SEQ ID NO:4) and 5′-CAGUUCA AUGGUGGGAGGGdTdT-3′ (SEQ ID NO:5). This Example follows cell proliferation of Stat3-ER-expressing A375 cells treated with 4HT or a combination of 4HT and OSM, and in the absence or in the presence of siRNA to TEL.

Protein analysis was carried out by Western blotting. In brief, cells were harvested in NP40 extraction buffer (50 mM Tris (pH 7.5), 1 mM EGTA, 5 mM EDTA, 120 mM NaCl, 1% NP40, 2 mM sodium orthovanadate, 50 mM sodium fluoride, 20 mM [symbol] -glycerophosphate, 10 μg/ml leupeptin, 10 μg/ml aprotinin, and 0.5 mM phenylmethylsulfonyl fluoride) for 5 min on ice. The lysates were clarified by centrifugation and protein concentrations were determined using the Bio-Rad protein assay reagent (Bio-Rad Laboratories Gmbh, Munich, Germany). For immunoprecipitaton, equal amounts of proteins were incubated with Stat3 (C-20) from Santa Cruz Biotechnology (Santa Cruz, Calif.) for 1 hr. Following SDS-polyacrylamide gel eletrophoresis (SDS-PAGE), proteins were blotted onto polyvinylidene difluoride membranes (Boehringer Mannheim, Mannheim, Germany). After blocking with 10% horse serum (Life Technologies, Inc.) in 50 mM Tris pH 7.5, 150 mM NaCl, 0.05% Tween 20, filters were probed with specific antibodies; c-TEL, Stat3 (C-20) from Santa Cruz Biotechnology (Santa Cruz, Calif.), phospho-Stat3 (Tyr 705) from Cell Signaling Technology (Beverly, Mass.). Proteins were visualized with peroxidase-coupled secondary antibodies using the enhanced chemiluminescence detection system (Amersham Pharmacia Biotech, Little Chalfont, United Kingdom).

Analysis at the protein level confirmed that TEL was strongly increased upon 4HT or 4HT/OSM treatment. This increase was largely prevented in the presence of TEL siRNA. Surprisingly, in the presence of TEL siRNA, Stat3-mediated inhibition of A375 cell proliferation was significantly increased (from about 28% to about 40%). These results indicated that TEL acts as a Stat3-induced negative regulator of Stat3 activity.

To test whether inhibition of TEL expression was directly affecting Stat3 activity, Stat3-dependent transcriptional activity was assayed in Stat3-ER-expressing A375 in the absence or the presence of TEL siRNA using the luciferase assay described above. Stat3-dependent transcription of luciferase, induced by 4HT or OSM, was significantly increased (from about 6 to 16 fold increase over control, and from about 9 fold to 18 fold increase over control respectively) when TEL expression was reduced by siRNA.

Therefore, expression of TEL was strongly decreased upon transfection of TEL siRNA relative to the levels observed in cells transfected with the control LacZ siRNA. In addition, the anti-proliferative effects of Stat3 in 4HT and OSM treated Stat3ER A375 cells were stronger in TEL siRNA-treated cultures in comparison to control LacZ siRNA treated cultures, further supporting TEL's function as a Stat3-induced negative regulator of Stat3 signaling.

Overexpression of TEL in Stat3ER A375 cells and in HEK293 cells resulted in a strong reduction in Stat3 activity in response to 4HT (50 to 75%) and to OSM (>70%). These results demonstrate that TEL is a negative regulator of Stat3 transcriptional activity.

In summary, the inhibition of breast carcinoma and melanoma cell proliferation by IL-6-type cytokines, as exemplified here with A375 cells, is dependent on Stat3 activity, which can be induced by reducing TEL activity.

### Example 7

### Inhibition of TEL Activity by Trichostatin A

TEL repressor activity is dependent on recruitment of a co-repressor complex, comprising mSin3A, NcoR and SMRT (Chakrabarti and Nucifora 1999; Wang and Hiebert 2001), which are known to interact with histone deacetylases (HDACs). This Example sets out to see whether HDAC recruitment might play a role in TEL-dependent repression of Stat3 transcriptional activity.

In brief, TEL- or control, pcDNA3.1-transfected Stat3ERA375 cells, treated with 4HT were cultured in the presence or absence of Trichostatin A (TSA; 250 nM), a general HDAC inhibitor, essentially as described above. Addition of TSA to 4HT-stimulated cells prevented the repression of Stat3 activity by TEL, but had no effects on Stat3 mediated transcriptional activity in pcDNA3.1-transfected cells. Thus, the inhibitory effect of TEL on Stat3-mediated transcription is dependent on the recruitment of HDACs.

### Example 8

### TEL Represses Stat3 Activity Independently of the DNA-Binding Domain

This Example examines which regions of TEL are required for repression of Stat3 transcriptional activity by expressing various TEL mutants in Stat3ER-A375 cells or HEK-293 cells and evaluating their effects on 4HT- or OSM-induced Stat3 transcriptional activity. Since the co-repressor, HDAC-containing complex is known to interact with at least two different domains of TEL, the pointed domain and a central repression domain (Chakrabarti and Nucifora 1999; Wang and Hiebert 2001) both these constructs were included in the assay. The constructs used were: TELΔ41-127 (i.e. ΔP), TELΔ122-176, TELΔ122-217, TELΔ268-333, TELΔ303-333, TELΔ333-352, TELΔ442-452, TELDBDM (R396K; R399K).

Briefly, Stat3-ER-A375 cells and HEK 293 cells were transfected with the different TEL mutants, a Stat3 reporter plasmid and a Renilla plasmid. Cells were treated with 4HT or OSM for 24 h and luciferase activity was measured using the luciferase assay described in Example 1. Only TEL ΔP, missing the pointed domain, failed to repress Stat3 activity. TEL delta 333-452 represses Stat3 activity, whereas TEL delta 442-452 is not able to repress Stat3 activity. TEL, TEL delta P, TEL delta 333-452 and to a less extent TEL delta 442-452 still interact with Stat3. The DNA-binding mutant of TEL retained the ability to repress 4HT- or OSM-induced Stat3 activity in both cell types, suggesting that TEL represses Stat3 activity independently of the DNA-binding domain but requires at least a portion of the pointed domain.

### Example 9

### TEL Interacts Directly with Stat3

Since the TEL DNA-binding domain is not required for repression of Stat3 activity, this Example tests the possibility that TEL interacts directly with Stat3. Extracts from nuclear-enriched fractions were prepared from control or OSM-stimulated A375 cells. Antibody probing nuclear lysates reveals the expected increase in Stat3 levels following OSM treatment, while the nuclear content of TEL is not affected by OSM. Endogenous TEL was present in Stat3 immunoprecipitates and the levels of TEL associating with Stat3 increased in OSM-treated compared to control-treated nuclear extracts. Conversely, Stat3 is present in a TEL-containing complex, pulled down through an immobilized GGM-containing oligonucleotide, which is a binding site for TEL. Taken together, these results suggest that TEL represses Stat3 transcriptional activity by interacting with Stat3 directly and recruiting HDACs to the Stat3 transcriptional complex.

All publications referred to herein, as well as U.S. patent application 60/495,309 filed Aug. 14, 2003 are hereby incorporated by reference as if each were referred to individually.

1. A method of identifying an agent effective in modulating Stat3-dependent cell proliferation, said method comprising the steps of:
i) incubating TEL/Etv6 with a compound;
ii) detecting TEL/Etv6 activity; and
iii) determining a compound-induced modulation in the TEL/Etv6 activity relative to when said compound is absent, wherein an alteration of the TEL/Etv6 activity in the presence of the compound is indicative of an agent effective in modulating Stat3-dependent cell proliferation.

2. The method according to claim 1, wherein said modulation is inhibition of TEI/Etv6 activity and said agent is effective in enhancing cytokine-induced inhibition of cell proliferation.

3. The method according to claim 1, wherein said modulation is activation of TEL/Etv6 activity and said agent is effective in inhibiting proliferation of cells expressing Stat 3, wherein said Stat3 is phosphorylated.

4. The method of claim 3, wherein said cell proliferation is independent of ras activity.

5. The method of the preceding claims, wherein said cell proliferation is of a melanoma or carcinoma.

6. A method for identifying an agent effective in modulating Stat3-dependent cell proliferation, said method comprising the steps of:
(i) incubating at least one TEUEtv6 polypeptide selected from the group consisting of TEL/Etv6, a variant and a fragment thereof, with a binding partner in the presence of a test compound; and
(ii) determining whether the presence of a test compound modulates the interaction between said TEL/Etv6 polypeptide and said binding partner relative to when said test compound is absent.

7. The method according to claim 6, wherein the variant or fragment of TEL/Etv6 has the ability to bind Stat3.

8. The method according to claim 6 wherein the fragment of TEL/Etv6 is between 50 and 350 amino acids in length.

9. The method according to claim 6, wherein said binding partner is Stats, a variant or fragment thereof.

10. The method of the preceding claims, further comprising confirming that the test compound is a modulator of Stat3-dependent cell proliferation.

11. The method according to claim 6, wherein said TEL/Etv6 polypeptide or the binding partner is labelled with a detectable label, and the other is immobilised on a solid support.

12. The method according to the preceding claim wherein the modulation is inhibition of said interaction.

13. The method according to claim 12 comprising the step of confirming that the substance inhibits cell proliferation of a cytokine-sensitive cancer.

14. The method according to claim 12, comprising determining whether said test compound inhibits the physical association between TEL/Etv6 and Stat3.

15. The method according to claim 6 said method comprising the steps of:
(i) contacting a cell expressing TEL/Etv6, a variant or fragment thereof which has the ability to interact with said binding partner, with a test compound, and
(ii) identifying substances which inhibit said interaction in said cell.

16. The method according to claim 15, said method comprising:
(i) providing a cell capable of expressing the TEL/Etv6 polypeptide and its binding partner and a reporter gene construct,
(ii) contacting the cell with a test compound,
whereby inhibition by the test compound of binding between the TEL/Etv6 polypeptide and the binding partner can be observed as a reduction of reporter gene expression.

17. A mammalian cell capable of expressing a TEL/Etv6 polypeptide, its binding partner, and a reporter gene construct, whereby binding between said TEL/Etv6 polypeptide and said binding partner can be observed by reporter gene expression.

18. A method of inhibiting Stat3 expressing cancer cell proliferation, said method comprising contacting a cancer cell expressing Stat3 with an effective amount of an activator of TEL in an amount sufficient to inhibit Stat3 activity.

19. The method of claim 18, wherein said Stat3 is phosphorylated.

20. A method of inhibiting cytokine sensitive cancers, said method comprising contacting a cytokine-sensitive cancer cell with an effective amount of an inhibitor of TEL activity in an amount sufficient to enhance Stat3 activity.

21. The method of claim 20, wherein the inhibition of activity is caused by down-regulating TEL/Etv6, or a homologue thereof in the cell.

22. The method of claim 21, wherein said down-regulation is caused by RNAi.

23. The method of claim 22, wherein said down-regulation is caused by an at least partially double-stranded RNA of between 20 and 25 bps in length, comprising an RNA sequence encoding a portion of TEL/Etv6 or a homologue thereof.

24. The method of claim 20, wherein said TEL inhibitor is an antibody or antibody fragment.

25. The method according to claim 20, wherein the inhibition of activity is caused by inhibiting the interaction of TEL/Etv6, or a homologue thereof with a binding partner in the cell.

26. The method according to claim 25 wherein the binding partner is Stat3.

27. Use of an at least partially double-stranded RNA comprising an RNA sequence encoding TEL/Etv6, a homologue or a fragment thereof, to inhibit cell proliferation of a cytokine-sensitive cancer cell.

28. Use of the double-stranded RNA according to in claim 27, wherein said dsRNA is an siRNA duplex of between 20 and 25 bps.

29. Use of an inhibitor of TEL/Etv6 activity in the preparation of a medicament for the treatment of a patient suffering from a cytokine-sensitive cancer.

30. Use of an activator of TEL/Etv6 activity in the preparation of a medicament for the treatment of a patient suffering from STAT3 expressing cancer.
